(19) United States
(12) Patent Application Publication
(10) Pub. No.: US 2025/0104801 A1
(43) Pub. Date: Mar. 27, 2025

(54) Method for Engineering Proteins

(71) Applicant: Kcat Enzymatic Private Limited, Bengaluru (IN)

(72) Inventors: Pravin Kumar R., Bengaluru (IN); Gladstone Sigamani G., Bengaluru (IN); Roopa L., Bengaluru (IN)

(73) Assignee: Kcat Enzymatic Private Limited, Bengaluru, KA (IN)

(21) Appl. No.: 18/501,060

(22) Filed: Nov. 3, 2023

(30) Foreign Application Priority Data
Sep. 25, 2023 (IN) 202341064088

(51) Int. Cl. G16B 15/00; G16B 20/50; G16B 30/10

(52) U.S. Cl. CPC G16B 15/00; G16B 20/50; G16B 30/10

(57) ABSTRACT

Presented herein is a computer-implemented method for protein engineering that constructs a three-dimensional gridspace around a protein. Different probes, simulating the interactions of amino acids are iteratively placed within the grid. Pair Interaction Energy is calculated using FMO technique for probe-protein interactions. An algorithmic process calculates the sum of PIEs, extending from each grid point to its neighbors, until a higher cumulative PIE is obtained. Grid points are then grouped into patches and an alignment process matches a derived query probe pattern from each patch against existing patterns of an internal database. Mutations are made based on the highest PIE probe-amino acid pairs from the matched pattern, leading to a modified protein. This process can be iterated for generating optimal variants and can be localized to any part of the protein for identification of patches. The invention covers a computer system and non-transitory computer-readable medium to implement the disclosed method.

### FIELD OF THE INVENTION

This invention relates to the field of Protein Engineering

### BACKGROUND OF THE INVENTION

Proteins are natural molecules synthesized by living organisms, function as vital catalysts for biochemical reactions within these life forms. They encompass a range of molecules, including enzymes and antibodies. While their potential has been used for commercial production across multiple industries, inherent limitations of wild-type proteins often curb their industrial effectiveness. These proteins, having evolved for specific biological contexts, might not exhibit the desired stability or functionality when exposed to the rigorous conditions of industrial processes, such as high temperatures, extreme pH variances, and aggressive chemicals. This instability can compromise their performance, leading to suboptimal catalytic or binding activities, especially when confronted with high substrate concentrations or unfamiliar environments.

Another obstacle with wild-type proteins for industrial applications is the complex scale-up of their production. Typically, production relies on specific host organisms or intricate protein expression systems. This dependency not only constrains the diversity of obtainable protein functionalities but also presents challenges in scalability. As a consequence, industries often lean towards synthetic chemistries for large-scale production. Such methods, although effective, come with their own set of challenges, especially concerning environmental impact. They often result in significant non-biodegradable waste, worsening ecological concerns.

The challenges above underscore the necessity of protein engineering, by using state-of-the-art techniques, it's possible to expand the realm of protein diversity, the creating novel proteins, ones that are tailor-made with enhanced functionalities, improved stability, and scalability. Protein engineering essentially reshapes the protein landscape, introducing molecules that are not just mimics of their wild-type counterparts but are optimized versions, primed for industrial applications.

However, this glowing potential of protein engineering is sometimes dimmed by the shortcomings of current methodologies. Traditional protein engineering methods often hinge on random mutagenesis approaches. Mutagenesis indeed involves introducing mutations in the DNA sequence. These changes in the DNA sequence can result in alterations in the corresponding protein products (Reetz, M. T., & Carballeira, J. D., 2007). Depending on the nature of the mutation (e.g., silent, missense, nonsense), this can lead to proteins that have different amino acid sequences and potentially altered functions or structures. In random mutagenesis, mutations are introduced without a specific target or design in mind. As a result, while you can obtain a diverse pool of protein variants, predicting which mutations will result in beneficial changes is challenging.

Directed evolution, another prevalent method, involves mimicking natural evolution in a lab setting by inducing genetic variations and then selecting proteins with desired traits (Bloom, J. D., & Arnold, F. H., 2009, Yuan L, et. al., 2005). Though inspired by nature, directed evolution requires multiple iterative rounds and a vast number of experiments, making the process resource-intensive. Such methods, despite their promise, might not always yield the most optimized protein variants, leaving room for inefficiencies.

Rational redesign typically leans on sequence homology to suggest amino acid substitutions. However, this method might overlook the intricate structural characteristics of proteins. On the other hand, while directed evolution can yield potent mutants, it's hindered by its inherently low-throughput nature and the pressing need for efficient assays to sift through a multitude of potential mutants. (Chen, R, 2001, You, L. & Arnold, F. H., 1996, Fox, R. J. et al, 2007). Contemporary protein engineering strategies predominantly utilize low-throughput screening methods that pinpoint the nuanced attributes of enzymes (Korkegian, A., et. al, 2005). The efficacy in generating compact but high-quality mutant libraries hinges on the diverse functionalities derived from protein sequences and the robustness of the screening and selection assays in isolating desired mutants. Several techniques have been brought to the fore to increase the success rate like In-depth computational analyses weighing the thermodynamic and spatial aspects of the enzyme-substrate interplay. Hands-on in vitro mutagenesis tests, Evaluating activity trends from preliminary directed evolution trials (Saraf, M. C., et. al, 2004).

Of late, advanced statistical methods have been deployed to map the relationship between protein sequences and their functions. These techniques streamline the evolutionary trajectory by swiftly zeroing in on advantageous genetic variability for recombination (Voigt, C. A. et. al., 2003). To further delve into and optimize enzymes, cutting-edge computational tools, such as molecular dynamics (MD) and quantum mechanics/molecular mechanics (QM/MM) methodologies, have been employed (Fox, R. et al., 2003, Warshel, A. & Levitt, M, 1976)

Clearly the need for a more precise, efficient, and holistic protein engineering strategy is evident, a strategy that doesn't just rely on chance mutations or labor-intensive experimental iterations but incorporates advanced computational tools, real-time feedback, and a nuanced understanding of protein structures and functions.

The present invention introduces a method for protein engineering using a 3D grid system. Within this grid, molecular probes representing amino acid side chains and solvent molecules interact with the target protein. The pair interaction energy between probes and protein amino acids is calculated using the fragment molecular orbital (FMO) method, which provides detailed insights into protein stability. Based on these energy calculations, unstable regions within the protein are identified. These regions are then strategically modified by introducing stable amino acids to enhance the protein's overall stability and functionality. The Fragment Molecular Orbital (FMO) method can tackle large molecular systems, such as proteins, by dissecting them into smaller, more manageable fragments for analysis, making it scalable for protein engineering applications. It provides a detailed quantum mechanical representation of interactions offering insights with high precision about the pair interaction energies between molecular probes and protein amino acids. By segmenting the protein into fragments, the FMO method can focus computational resources more efficiently, saving time and computational costs compared to analyzing the entire protein as a single entity. The method points out specific amino acid residues or regions in the protein that significantly contribute to its stability or function. The results obtained from FMO can be seamlessly integrated with other computational methods, like molecular dynamics simulations or machine learning algorithms, to provide a holistic approach to protein engineering. With the insights provided about the nature and strength of interactions at the molecular level, protein engineering can be more directed and strategic making informed decisions on which residues to mutate and predict the effects of these mutations with higher confidence. It helps in ensuring that the designed or engineered protein not only has the desired function but also maintains stability and viability in its intended application. Top of Form

The Pair Interaction Energy quantifies the strength and nature of interactions between two fragments, such as an amino acid residue and a molecular probe and provides a deeper understanding of how specific residues contribute to the protein's overall stability or activity while the FMO method offers a granular understanding of these interactions. Neural networks are employed to provide the iterative optimization, ensuring that the engineering process is both precise and efficient.

### Objects of the Invention

The primary objective of the invention is a method to engineer proteins for enhanced characteristics, tailoring them to perform specific functions or adapt to specific conditions. Through engineering, wild-type proteins can be modified to have increased stability, enhanced activity, broader substrate specificity, improved binding affinity, higher catalytic turnover, and greater resistance to denaturation, to name a few. The art of protein engineering lies in understanding and manipulating the intricate interactions of amino acid side chains within the protein structure.

To achieve this, the proposed method uses a 3D grid system made of gridpoints, with each grid point spaced precisely 1 Å apart. At these grid points, molecular probes representing the side chains of amino acids and solvent molecules are iteratively place on the gridpoints. The protein to be engineered is placed in the grid, where the probes interact with the amino acids of the protein. The pair interaction energy is calculated using the fragment molecular orbital (FMO) method which provides vital insights into these interactions. The FMO method explores deep into the quantum mechanics of the protein, breaking it down into manageable fragments, thereby offering a precise understanding of the interactions between the probes and the amino acids.

Analyzing these energy dynamics helps identify regions of stability or vulnerability within the protein. By identifying regions with higher energy (hence lower stability), strategic substitutions can be made across the protein structure. Amino acids known for their stability in various protein environments, particularly those with low pair interaction energies are introduced at these points. The result is a fortified protein, its weaknesses meticulously addressed and turned into strengths.

A Neural Network (NN) algorithm is employed which recognizes patterns, learn from data, and make informed decisions, making them exceptionally suited for the iterative process of selecting the best mutations. Through multiple iterations, the algorithm continually refines the protein, ensuring the most optimized version emerges.

The grid-based approach has the ability to precisely discern and analyze these energetic interactions. Such an analysis offers insights into the energetic consequences of potential amino acid substitutions or mutations. By evaluating these energy parameters, we can predict the repercussions of specific amino acid changes on the overall stability of the protein.

Building upon this foundation, the invention aims to harness this knowledge to strategically design protein variants with enhanced characteristics, catering to an array of commercial applications. Such engineered proteins may be equipped with heightened catalytic ability, enabling them to facilitate chemical reactions more efficiently. Furthermore, modifications can also improve their affinity for binding to specific molecules, opening doors to innovative applications in sectors like pharmaceuticals, biotechnology, agriculture, biomaterials drug delivery and molecular sensing. By achieving this, the invention aims to bridge the gap between theoretical protein design and real-world industrial applications with a new era of bioengineering solutions.

### Harnessing the Fragment Molecular Orbital Method for Advanced Protein Engineering

The overarching aim of this invention revolves around the conceptualization and subsequent implementation of a computational methodology designed to elevate the standards of protein engineering. The methodology harnesses power of the Fragment Molecular Orbital (FMO) method, a quantum mechanical approach that has shown great promise in the realm of molecular modeling and simulation (Kitaura, K., et al., 2002)

Proteins, by virtue of their various roles in biological systems, have been at the forefront of biochemical research. These macromolecules have been extensively tailored and modified over the years to meet specific requirements, be it in medical therapeutics, industrial catalysis, or environmental bioremediation. However, the challenge has perennially been to identify precise regions within these proteins that are amenable to modifications without compromising their structural integrity or functional efficacy (Arnold, F. H, 2018).

Addressing this problem, our proposed methodology anchors its foundation on the FMO method. Rooted in the principles of quantum mechanics, the FMO approach dissects extensive molecular systems into smaller, more manageable units termed ‘fragments’ (Fedorov, D. G., et al., 2012). These fragments, depending on the context and complexity, can span the range from being single atoms to entire amino acid residues.

What distinguishes the FMO method from conventional quantum mechanical approaches is its ability to compute the electronic structure of each fragment independently, and then in conjunction, leading to a holistic representation of the molecule in question. Mathematically, the total energy of the system is envisaged as a cumulative sum of the energies of individual fragments and their interactions (Nakano, T., et al, 2002)

$${E}_{total} = {\sum }_{i} {E}_{i} + {\sum }_{i < j} {E}_{ij}^{( 2 )}$$

Here, Ei demarcates the energy of fragment i, and Eij (2) encapsulates the pairwise interaction energy between fragments i and j. This pairwise interaction energy, pivotal to decoding the molecular narrative, is elucidated as:

$${E}_{ij}^{( 2 )} = {E}_{ij} - {E}_{i} - {E}_{j}$$

Flexibility remains at the core of the FMO method. The approach accommodates varying basis sets, ranging from minimal to extensive, based on the computational ability and the granularity of insights desired. To heighten the fidelity of predictions, electron correlation methodologies like MP2 or CCSD can be seamlessly incorporated within the FMO framework (Andrzej M. Oleś., et al., 1986). Recognizing the profound influence of external conditions, especially solvation effects, our method integrates the Polarizable Continuum Model (PCM), ensuring an actual representation of solvent-induced perturbations (Mennucci, B., 2012).

The computational efficiency inherent to the FMO methodology ensures, each fragment, being independently operable, lends itself to parallel processing, which, when leveraged aptly, can lead to monumental reductions in computational times (Y. Mochizuki., et al., 2008).

In the specific context of protein engineering, the applicability and impact of the FMO method are nothing short of transformative. The approach furnishes granular insights into the individual energy contributions of amino acids, particularly in scenarios involving protein-substrate or protein-ligand interactions. Armed with this quantum-derived wisdom, the proposed methodology meticulously singles out regions within proteins characterized by low-energy and high stability. These regions subsequently undergo strategic substitutions with amino acids, ubiquitously present across protein families. As a result, proteins that are not only structurally robust but are also functionally enhanced and optimized for diverse applications are designed (Bloom, J. D., et al., 2005).

Furthermore, the nexus between the FMO approach and machine learning algorithms, particularly Neural Networks (NN), strengthens the efficacy of the method. The synergy facilitates iterative fine-tuning of amino acid substitutions, leading to an optimized protein design (Ragoza, M., et al.).

In summation, this invention, deeply rooted in the principles of the FMO method, seeks to revolutionize protein engineering. It's a union of quantum mechanics, biology, and computational intelligence, and promises to sculpt proteins tailored to perfection for a range of applications.

### SUMMARY OF THE INVENTION

Protein engineering involves modifying proteins to improve their performance, stability, specificity, or other desired properties. One approach is to target specific residues on the protein that correspond to high-energy regions that are often associated with important functional sites or are areas that undergo conformational changes during protein interactions. The present invention describes a computer-based method for protein engineering using Pair Interaction Energy. The method allows the amino acids of a protein to interact with different types of biomolecular probes within a grid and calculates the PIE for each interaction.

A pattern representing the interactions and their associated PIE values is derived. This pattern, known as the query probe pattern, is then compared to an internal database probe pattern. The regions of the protein encoded in the pattern that exhibit high PIE values are identified as low stability regions and are mutated using amino acids from an internally developed protein database. The proteins in the internal database have undergone amino acid-probe PIE calculations, generating internal database probe patterns and their associated PIE values.

The amino acids for mutations are selected based on matching regions of the protein of interest, corresponding to the query probe pattern, and the proteins in the internal database corresponding to the internal probe database pattern. This matching is accomplished using an alignment algorithm. The method involves exploring all possible amino acid substitutions at specific positions in the 3D structure of the protein, considering various permutations and combinations, for one query probe pattern that might include 7 to 10 amino acids of a protein, thousands of matching internal database probe pattern may arise. However, the algorithm runs PIE calculations on every variant segment obtained from mutations with all the matching internal probe pattern, to arrive at the most suitable mutation. Through an iterative process, specific positions on the protein are determined for mutation, and the best possible variants with 2 to 8 amino acid substitutions are generated. This method allows for the exploration of a large sequence space on proteins and enables rapid transformation to a structural space with desirable properties.

Pair interaction energy plays a crucial role in understanding the structure, stability, and function of biological systems. In proteins these interactions are primarily electrostatic in nature, arising from the charges on the amino acids' side chains. These interactions can be attractive or repulsive, influencing the folding and stability of proteins. The pair interaction energies between amino acids within a protein determine the protein's three-dimensional structure, which, in turn, dictates its stability for a desired function.

By employing this method, materials, resources, and time can be saved in the development and engineering of proteins with desired or improved properties. The present invention employs Neural networks to recognize patterns to extract meaningful information from complex datasets. The network is trained to recognize patterns within the protein sequences to identifying critical regions, predicting protein properties, or making decisions regarding mutations. Also, the algorithm is trained in an iterative manner, allowing for continuous improvement and refinement of the protein engineering process. Feedback from experimental results or additional data can be incorporated to update the neural network and enhance its performance over time. This adaptability makes the algorithm a valuable tool for accelerating and streamlining the protein engineering workflow.

Protein engineering stands at the node of biology and technology, with the objective of reshaping proteins to optimize their functionality, stability, specificity, and various other properties. At the heart of this scientific endeavor is the quest to identify and target high-energy regions of proteins-sites that are often integral to their function or undergo substantial conformational shifts during interactions.

The core of the present invention is a computer-assisted method for protein engineering anchored on the principle of Pair Interaction Energy (PIE) using fragment molecular orbitals (FMO). This method visualizes a protein's amino acids within a precise three-dimensional grid, allowing them to engage in interactions with biomolecular probes. Every single interaction is quantified using PIE, leading to the generation of a unique query probe pattern—an insightful blueprint that encapsulates the interactions and their respective PIE values.

Equipped with this query pattern, the next step entails a comparative analysis against an internal database teeming with pre-calculated probe patterns. PIE values act as reliable indicators of stability; higher PIE values typically highlight regions of lesser stability. Recognizing these regions, the protein is subject to strategic mutations. Amino acids enlisted for these mutations are sourced from an exhaustive internal database of proteins that have already been subjected to amino acid-probe PIE evaluations.

The method explores an expansive sequence space within proteins. For a single query probe pattern encompassing just 7-10 amino acids, a staggering array of matching internal database probe patterns emerge. Yet, the inherent efficiency of the accompanying algorithm ensures that every potential mutation is probed, guaranteeing an optimal selection.

The importance of Pair Interaction Energy in the realm of biological systems is highly significant, predominantly electrostatic; these interactions stem from charges present on amino acid side chains. Whether they attract or repel, they sculpt the protein's architecture, in turn governing its stability and function. With this information, the invention not only accelerates the protein engineering trajectory but also ensures resource optimization, culminating in proteins precisely tailored for specific applications.

Furthermore, the invention embraces the power of Neural Networks, elevating its capabilities to new horizons. These networks have been trained to decipher patterns within protein sequences, identifying pivotal regions, forecasting protein behaviors, and guiding mutation decisions. An iterative training regimen ensures that the algorithm remains dynamic, continually assimilating feedback, and refining its ability. As a proof to its adaptability, the algorithm seamlessly integrates experimental results, perpetually improving its efficiency and accuracy. In essence, this invention provides a streamlined, adaptable, and highly efficient avenue for the next frontier of protein engineering.

### BRIEF DESCRIPTION OF DRAWINGS

FIG. 1. The structural representation of 2D structures of the 20 natural amino acids that are modified by excluding backbone, water, and dimethyl sulfoxide (DMSO). Each structure features its unique alpha carbon (highlighted in bold), depicted as a heavier atom label. The probes, designated by their chemical structures, are arranged in a grid for easy comparison and identification. This visualization aids in understanding the chemical nature and potential interaction sites of each probe within the grid. These probes represent the side chains of specific amino acids. Each amino acid has a unique side chain that contributes to its properties and interactions with other molecules. Here, the single-letter codes of amino acids (e.g., A, C, D, E etc.,) indicate specific amino acids. For instance, As represents the side chain of Alanine, Cs represents the side chain of Cysteine and so on. Amino acid side chains are essential determinants of a protein's structure and function. They can be polar, non-polar, charged (positive or negative), or aromatic. Each side chain has distinct chemical properties that dictate its interactions within the protein and with other molecules. The probes Was and Dms are Solvent Probes. These probes represent common solvents used in biochemical experiments. Was probe is water Water is a polar solvent. It can form hydrogen bonds and is involved in maintaining the structure and function of many biological molecules, including proteins. It plays a role in solvating ions and other polar molecules. Dms probe is Dimethyl Sulfoxide (DMSO). DMSO is a polar aprotic solvent. It can dissolve both polar and non-polar compounds. In biological settings, it's often used to enhance the penetration of molecules through biological membranes. When employed in the described method, these probes are essential in understanding the nature of interactions between the protein and its environment. By gauging the interaction energy between a protein's amino acid residues and these probes, insights can be derived about the protein's stability, affinity, and overall behavior in various conditions

FIG. 2. These matrices are typically constructed based on large datasets of aligned protein sequences. The idea is to calculate how often specific amino acid substitutions occur in evolutionarily related proteins. Each cell in the matrix represents a score or penalty for substituting one amino acid with another. The score can be positive (indicating similarity) or negative (indicating dissimilarity). The higher the score, the more similar the amino acids are considered. The diagonal cells represent matches between identical amino acids. These cells typically have the highest positive scores, as identical amino acids are considered highly similar. The off-diagonal cells represent substitutions between different amino acids. The values in these cells are based on the frequency of observed substitutions in the dataset used to construct the matrix. If a substitution is common, it will have a higher positive score, indicating similarity. If it is rare, it may have a lower score or even a negative score, indicating dissimilarity. To score an alignment between two protein sequences, you traverse the alignment and look up the scores for each pair of aligned amino acids and apply gap penalties for any gaps introduced. The total alignment score is the sum of these individual scores.

FIG. 3. (A.) The Grid of Alanine Side chain (As): This grid comprises probes positioned at each grid point by aligning the geometric center of the probes to each grid point. (B.) The Grid of Water (Was): This grid follows the same configuration method as the Alanine grid. The probes in each grid are fine-tuned to accommodate the protein's structure as elaborated in the detailed description. Fragment Molecular Orbital (FMO) energy and pair interaction energy are computed for each amino acid present in the protein and for each probe individually. The calculated energy is then registered at the corresponding grid point.

FIG. 4. The figure illustrates the fragment localized molecular orbitals involved in the interaction between LEU18 of an enzyme and the probe As. These molecular orbitals represent the electronic structure and play a key role in determining the nature of the interaction. The calculated pair interaction energy between LEU18 and As is-6.6 kcal/mol, indicating a strong attractive interaction. The PIE is composed of various terms: ES (Electrostatic Interaction) −2.1 kcal/mol, CT+J (Charge Transfer and Polarization Energy) −1.2 kcal/mol, DI (Dispersion Interaction) −0.8 kcal/mol, Ex (Exchange Interaction) 0.1 kcal/mol, and PCM (sol) (Polarizable Continuum Model Solvation Energy) −2.6 kcal/mol. These terms represent specific energy contributions to the overall PIE, highlighting the various forces involved in the interaction.

FIG. 5. The figure illustrates a section of the enzyme, ensconced within a grid populated by grid points and probes placed 2 Å apart. For clarity and convenience, only two types of probes are displayed from a mixed grid: one representative of water and the other symbolizing Alanine. These probes are strategically located close to Leucine 18 and their positions are determined based on the Fragment Molecular Orbital (FMO) and pair interaction energies calculations between Leucine 18 and the other amino acids. In this particular scenario, the geometric center of the probes is situated on the grid. The probes are then fine-tuned to achieve a Vanderwaals fit with the protein sidechain.

FIG. 6. SDS PAGE Gel shows the expression and isolation of Tyrosine Hydroxylase variant enzymes. Dark bands show induction of variants of Tyrosine Hydroxylase

FIG. 7. Reaction catalysed by enzyme Tyrosine Hydroxylase (Tyrosinase). L-Tyrosine is converted to L-DOPA

In FIGS. 8A-8D the depicted HPLC chromatograms offer an insightful representation of the enzymatic conversion of L-Tyrosine to L-DOPA. This visual distinction clearly delineates the activity of A) the wild-type enzyme against the performances of the engineered enzyme variants shown in B, C, and D. The assay, designed to emulate optimal catalytic conditions for these enzymes, was executed under the following parameters: a substrate (Tyrosine) concentration of 10 mg/ml; an alkaline environment with a pH of 9.5; a temperature maintained at 45° C.; and an enzyme concentration of 0.1 mg/ml. The entire reaction spaned a 45-minute timeframe. From the chromatograms, an evident observation emerges: the wild enzyme largely remains dormant, presenting no discernible activity within the given conditions. In contrast, the engineered variants demonstrated substantial conversion. This conspicuous difference emphasizes the advancements achieved through the engineering process. Notably, the enzyme load was just 1% for all the complete conversion by the engineered enzymes. This observation is particularly promising, highlighting the efficacy of the engineered enzymes and hinting at potential cost savings in large-scale applications.

### DEFINITIONS OR TERMINOLOGIES

Unless explicitly defined otherwise in this document, all technical and scientific terms utilized herein are presumed to hold the same significance as commonly interpreted by individuals possessing average expertise in the relevant field. Numerous widely recognized scientific dictionaries that describe the terms used herein are readily accessible to those possessing expertise in the respective field. Any method and material similar or equivalent to those described in this document find use in the practice of the embodiments described in this document. Terms defined immediately below will be more fully understood by reference to the specification as a whole. The provided definitions serve the purpose of clarifying specific embodiments and enhancing the comprehension of intricate concepts expounded upon in this specification. It's important to note that these definitions are not intended to curtail the complete extent of the information presented in this disclosure. Specifically, it should be understood that this description is not limited to the particular proteins, probes or systems described herein, as these may vary, depending on the context in which they are used by those skilled in the art.

Protein of Interest refers to the protein to be engineered, that is in free (apo) or complexed with a small molecule, can refer to any conformation of the same derived from transitional conformational changes obtained from a MD or QM/MM simulation. Here a query can be the whole protein of interest or a region of the protein which is to be engineered.

### Representative Protein Structures

Representative Protein Structures refer to a representative from various classes of proteins as available which will stand as an example to the structural and functional features specific to that class of the protein

### Fragment

Any region on the protein, ideally group of amino acids of the protein

### Grid

A grid in this context is a 3-Dimensional space that is made of grid points spaced 1 Å from one another and of size big enough to accommodate the protein of interest. At every grid point is placed a probe which interact with an amino acid of the protein, for which a pair interaction energy is calculated. Every grid point that falls within 1 Å of any amino acid of the protein or is considered for a calculation.

### Probes

We describe probes as molecules that will interact with the aminoacid residues of the protein. For every probe interacting with an amino acid, the pair interaction energies of the atoms are calculated.

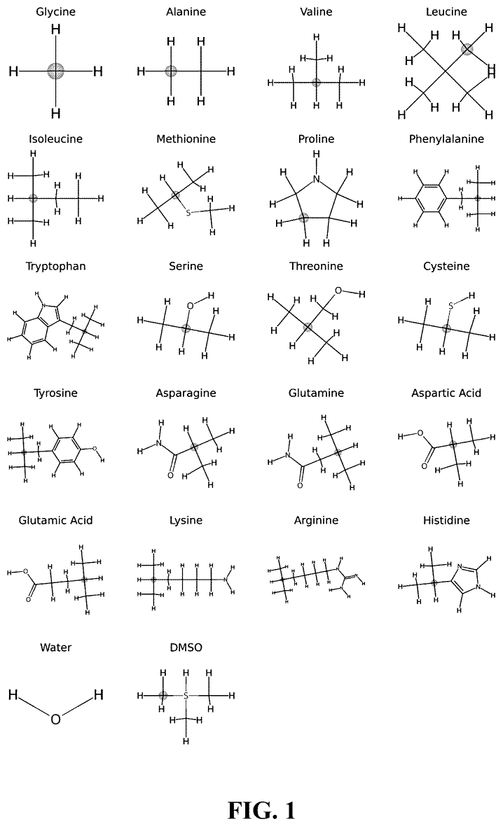

These probes represent the side chains of specific amino acids. Each amino acid has a unique side chain that contributes to its properties and interactions with other molecules. Here, the single-letter codes of amino acids (e.g., A, C, D, E etc.,) indicate specific amino acids. For instance, As represents the side chain of Alanine, Cs represents the side chain of Cysteine and so on. Amino acid side chains are essential determinants of a protein's structure and function. They can be polar, non-polar, charged (positive or negative), or aromatic. Each side chain has distinct chemical properties that dictate its interactions within the protein and with other molecules. The probes Was and Dms are Solvent Probes. These probes represent common solvents used in biochemical experiments. Was probe being water, water is a polar solvent. It can form hydrogen bonds and is involved in maintaining the structure and function of many biological molecules, including proteins. It plays a role in solvating ions and other polar molecules. Dms probe is Dimethyl Sulfoxide (DMSO). DMSO is a polar aprotic solvent. It can dissolve both polar and non-polar compounds. In biological settings, it's often used to enhance the penetration of molecules through biological membranes. When employed in the described method, these probes are essential in understanding the nature of interactions between the protein and its environment. By gauging the interaction energy between a protein's amino acid residues and these probes, insights can be derived about the protein's stability, affinity, and overall behavior in various conditions. Different types of probes employed in the method are described below with abbreviations and shown in FIG. 1.
Water—Was
DMSO-Dms-dimethylsufoxide which is a cosolvent in some enzymatic reactions
Side chain of Alanine—As
Side chain of Glycine—Gs
Side chain of Valine—Vs
Side chain of Leucine—Ls
Side chain of Isoleucine—Is
Side chain of Serine—Ss
Side chain of Threonine—Ts
Side chain of Methionine—Ms
Side chain of Cysteine—Cs
Side chain of Proline—Ps
Side chain of Phenylalanine—Fs
Side chain of Tyrosine—Ys
Side chain of Tryptophan—Ws
Side chain of Aspartic acid—Ds
Side chain of Glutamic acid—Es
Side chain of Aspargine—Ns
Side chain of Glutamine—Qs
Side chain of Histidine—Hs
Side chain of Lysine—Ks
Side chain of Arginine—Rs

The structural representation of 2D structures of the 20 natural amino acids that are modified by excluding backbone, water, and dimethyl sulfoxide (DMSO) are shown in FIG. 1. Each structure features its unique alpha carbon (highlighted in bold), depicted as a heavier atom label. The probes, designated by their chemical structures, are arranged in a grid for easy comparison and identification. This visualization aids in understanding the chemical nature and potential interaction sites of each probe within the grid. These probes represent the side chains of specific amino acids. Each amino acid has a unique side chain that contributes to its properties and interactions with other molecules. Here, the single-letter codes of amino acids (e.g., A, C, D, E etc.,) indicate specific amino acids. For instance, As represents the side chain of Alanine, Cs represents the side chain of Cysteine and so on. Amino acid side chains are essential determinants of a protein's structure and function. They can be polar, non-polar, charged (positive or negative), or aromatic. Each side chain has distinct chemical properties that dictate its interactions within the protein and with other molecules. The probes Was and Dms are Solvent Probes. These probes represent common solvents used in biochemical experiments. Was probe is water Water is a polar solvent. It can form hydrogen bonds and is involved in maintaining the structure and function of many biological molecules, including proteins. It plays a role in solvating ions and other polar molecules. Dms probe is Dimethyl Sulfoxide (DMSO). DMSO is a polar aprotic solvent. It can dissolve both polar and non-polar compounds. In biological settings, it's often used to enhance the penetration of molecules through biological membranes. When employed in the described method, these probes are essential in understanding the nature of interactions between the protein and its environment. By gauging the interaction energy between a protein's amino acid residues and these probes, insights can be derived about the protein's stability, affinity, and overall behavior in various conditions

### Molecular Orbital

A molecular orbital is a mathematical function that describes the behavior of electrons in a molecule. When atoms combine to form a molecule, their atomic orbitals overlap and combine to form molecular orbitals.

Molecular orbitals can be formed through two types of interactions: constructive and destructive. Constructive interactions lead to bonding molecular orbitals, which have lower energy than the atomic orbitals from which they were formed. Destructive interactions lead to antibonding molecular orbitals, which have higher energy than the atomic orbitals.

Molecular orbitals play a crucial role in determining the electronic structure and properties of molecules, such as their stability, reactivity, and spectroscopic properties. Therefore, molecular orbital theory is an essential tool for understanding chemical bonding and reactions in organic inorganic chemistry and biology

### FMO Method

The Fragment Molecular Orbital (FMO) method is a quantum chemical method that allows the accurate calculation of the electronic structure and properties of large molecular systems by dividing the system into smaller fragments and treating them separately. The FMO method uses the concept of pair interaction energy to calculate the interaction energy between the fragments.

In the FMO method, the molecular system is divided into smaller fragments, each of which is treated as a separate entity. The electron density of each fragment is described by its own set of molecular orbitals. The interaction between the fragments is then described by the PIE, which is the energy change that occurs when two fragments are brought together to form a complex.

The FMO method allows for the accurate calculation of the electronic structure and properties of large molecular systems by treating them as a collection of smaller fragments. The PIE is a key concept in the FMO method as it allows for the calculation of the interaction energy between the fragments.

### Pair Interaction Energy (PIE)

Pair interaction energy is the contribution to the total energy that is caused by an interaction between the objects being considered. The interaction energy usually depends on the relative position of the objects. Interaction energy between molecules A and B (ΔEAB) is determined as the difference between the energy of the dimer (EA, B) and the sum of the monomer energies (EA+EB).

$$Δ EAB = ( EAB ) - ( EA + EB ) .$$

The lower the pair interaction energy higher is the stability of the interaction.

Pair interaction energy is closely linked to molecular orbitals, as the energy of a molecular orbital is determined by the interactions between electrons in the molecule. When two atoms come together to form a molecule, their atomic orbitals overlap to create new molecular orbitals. The energy of these molecular orbitals is determined by the energies of the atomic orbitals and the interactions between electrons in the molecule.

The pair interaction energy, or the energy required to remove or add an electron to a molecular orbital, is related to the stability and reactivity of a molecule. If a molecular orbital has a low pair interaction energy, it is more stable and less reactive, since it requires less energy to remove or add an electron to the orbital. Conversely, if a molecular orbital has a high pair interaction energy, it is less stable and more reactive, since it requires more energy to remove or add an electron to the orbital.

In molecular orbital theory, the energies and shapes of molecular orbitals are calculated using quantum mechanical principles and can be used to predict the properties of molecules and their reactions. Therefore, the study of molecular orbitals and their pair interaction energies is essential for understanding the behaviour of molecules in chemistry and biology.

PIE is a measure of the strength of the interaction between two atoms in a molecule. The PIE is related to the molecular orbital theory through the concept of bond order.

Bond order is a measure of the number of electron pairs shared between two atoms in a molecule. In molecular orbital theory, bond order is calculated as the difference between the number of electrons in bonding molecular orbitals and the number of electrons in antibonding molecular orbitals, divided by two.

Bond order is directly related to pair interaction energy. When bond order increases, the pair interaction energy becomes more negative, indicating a stronger bond. Conversely, when bond order decreases, the pair interaction energy becomes less negative, indicating a weaker bond.

Pair Interaction Energy Calculation using Fragment Molecular Orbital Method. The Fragment Molecular Orbital method is a computational method used to calculate the electronic structure and properties of large molecular systems. The FMO method uses the concept of pair interaction energy to divide a large molecule into smaller fragments or subunits.

In the FMO method, the molecular system is partitioned into fragments, and the electronic structure of each fragment is calculated separately using molecular orbital theory. The total energy of the system is then calculated as the sum of the energies of the individual fragments plus the pair interaction energies between the fragments.

The pair interaction energy in the FMO method is calculated by comparing the energy of the system when the fragments are separated (infinite distance) with the energy of the system when the fragments are close together. The difference between these two energies is the pair interaction energy.

The FMO method is particularly useful for studying large molecular systems, such as proteins and polymers, where traditional quantum mechanical calculations c become computationally impractical. The FMO method provides a way to divide the system into smaller fragments that can be treated separately, thereby reducing the computational cost.

The FMO method is closely related to the concept of pair interaction energy because the calculation of the pair interaction energy is a key component of the FMO method. By using the FMO method to calculate the pair interaction energy between fragments, it is possible to obtain an accurate estimate of the electronic structure and properties of large molecular systems.

### Neural Network Algorithm

In the present embodiment neural network (NN) algorithm is used to guide the decision-making for improving the process for optimal engineering. The process of engineering is clearly defined with the desired outcomes and the criteria that makes a substitution the most suitable. The NN algorithm collects historical data from the process, including input parameters/settings and the corresponding outcomes and makes sure to also include information about which outcomes were considered good based on your criteria. The NN architecture takes input parameters/settings as input and produces an output indicating whether the outcome is good or not. This could be a binary classification task and the NN is trained using historical data, optimizing it to predict good outcomes based on select criteria. The trained NN is integrated into the optimization process. Here's how the process might work iteratively: Run the process with specific input parameters/settings, use the trained neural network to predict whether the outcome is good or not, if the outcome is not good according to the neural network's prediction, adjust the input parameters/settings, repeat the process until the neural network predicts a good outcome or until a maximum number of iterations is reached. There is feedback loop as the optimization process is run, new data about the outcomes and the input parameters/settings adjusted are collected. This data can be used to retrain the NN periodically, improving its ability to predict good outcomes based on select criteria.

This approach combines the predictive power of neural networks with an optimization loop to iteratively guide the decision-making process towards achieving the best results according to defined criteria.

Internal Database and Internal Database Probe Pattern. The invention involves
Retrieval of proteins from open-source databases, that represent different protein families and are classified based on various criteria such as structure, function, conserved domains and evolutionary relationships. A collection of such proteins to make an inhouse database is termed internal database. These proteins are naturally occurring stable structures, each performing a specific function in nature. Some examples of the internal database proteins include
Collagen: Collagen is the most abundant protein in the human body and a major component of connective tissues, such as skin, bones, tendons, and ligaments. It provides strength and structural support to these tissues.
Keratin: Keratin is a fibrous protein found in the epidermis (outer layer) of the skin, hair, nails, and feathers. It provides protection and structural integrity to these structures.
Actin and Myosin: Actin and myosin are proteins found in muscle cells. They are responsible for muscle contraction and play a crucial role in movement and locomotion.
Tubulin: Tubulin is a protein that makes up microtubules, which are essential components of the cytoskeleton. Microtubules provide structural support and are involved in intracellular transport and cell division.
Elastin: Elastin is a protein found in elastic tissues like the skin, arteries, and lungs. It imparts elasticity and allows tissues to stretch
Fibrin: Fibrin is a protein involved in blood clotting. It forms a mesh-like network during the coagulation process, which helps stop bleeding and promotes wound healing.
Laminins: Laminins are a family of proteins found in the extracellular matrix of tissues. They play a crucial role in cell adhesion, cell migration, and tissue organization.
Histones: Histones are proteins that help package and condense DNA into chromatin within the cell nucleus. They play a role in gene regulation and maintaining the structure of chromosomes.
Myoglobin: Myoglobin is a protein found in muscle cells that stores and transports oxygen within muscle tissues. It facilitates oxygen diffusion in muscles during periods of increased demand.
Troponin: Troponin is a complex of three proteins found in muscle cells, particularly in cardiac and skeletal muscles. It regulates muscle contraction by interacting with actin and tropomyosin.
Enzymes: Enzymes are a diverse group of proteins that catalyze biochemical reactions in the body. They act as biological catalysts, accelerating chemical reactions to maintain cellular processes and metabolism.
Hemoglobin: Hemoglobin is a protein found in red blood cells that binds to oxygen in the lungs and transports it to tissues throughout the body. It plays a crucial role in oxygen transportation.
Insulin: Insulin is a peptide hormone produced by the pancreas. It regulates blood glucose levels by facilitating the uptake of glucose by cells and inhibiting glucose production in the liver.
Antibodies: Antibodies, also known as immunoglobulins, are proteins produced by the immune system in response to foreign substances (antigens). They recognize and neutralize pathogens, thereby protecting the body from infections.
Hormones: Various hormones in the body are proteins or peptide-based molecules. Examples include growth hormone (GH), thyroid-stimulating hormone (TSH), and insulin-like growth factors (IGFs), which regulate growth and metabolism.
DNA Polymerase: DNA polymerase is an enzyme responsible for synthesizing new strands of DNA during replication and repair processes. It ensures accurate copying of the genetic information.
RNA Polymerase: RNA polymerase is an enzyme that catalyzes the transcription of DNA into RNA molecules. It is a key player in gene expression and the formation of various types of RNA.
Rhodopsin: Rhodopsin is a protein found in the light-sensitive cells (rods) of the retina. It is responsible for capturing light and initiating the visual signal transduction cascade.
Aquaporins: Aquaporins are a group of membrane proteins that facilitate the transport of water and other small molecules across cell membranes. They play a critical role in maintaining water balance in cells and tissues.
Growth Factors: Growth factors are signaling proteins that regulate cell growth, proliferation, and differentiation. They are involved in tissue repair, embryonic development, and wound healing.
   Each of the retrieved proteins undergoes a series of steps within the invention
   Each protein is placed in a 3D grid made of grid points spaced 1 Å apart (or 2 Å apart)
   Within the gridspace, at every gridpoint variety of probes are placed iteratively where each probe molecule simulates the interaction characteristics of an amino acid or a solvent molecule with the protein that is enclosed in the grid.
   The probe is placed such that, the geometric center of mass of the probe aligns with the center of the designated grid points. This ensures a standardized and consistent placement of the probes within the system, facilitating a systematic exploration of potential interactions across the entire grid.
   The probes are rotated to explore possible interactions with the protein of interest in the grid, however masked from one another to avoid inter-probe interactions The van der Waals (VDW) radius of the probe is adjusted to facilitate interactions, and the interaction energies are calculated using Pair Interaction Energy Decomposition Analysis (PIEDA) of the Fragment Molecular Orbital method. The energy of interaction is then stored at the respective grid points.
   The PIEs are calculated for each interaction across all grids, resulting in a comprehensive interaction profile for the protein.
   Each grid point stores energy details and the probe type that exhibited the least PIE during interactions with the protein's amino acids
   Subsequently, the grid now comprises of mixed probe types based on the ones that exhibited the least PIE on interaction with the amino acids of the protein in the grid.
   For example, it may be that aminoacid 23 of a protein shows the least PIE on interacting with the probe type As, among all the probes in the grid and amino acid 24 shows the least PIE on interacting with the probe type Fs, among all the probes, the new grid will have probe type As on the gridpoint interacting with amino acid 23 and the probe type Fs on the gridpoint interacting with amino acid 24.
   The computational process is extended by systematically augmenting the sum of the Pair Interaction Energy stored on each grid point, on the negative scale between each grid point and its neighboring grid points within the constructed three-dimensional grid space encapsulating the protein structure. This methodical progression persists until the integration of an additional grid point leads to a shift in the cumulative PIE towards the positive scale, signaling a less favorable interaction.
   Extension is done on every gridpoint, the extension can be explained as, a grid point being connected to its neighbour and the PIE stored on the neighbour is added to its PIE, if the cumulative PIE is lesser than the last PIE, then the connection grows to the next neighbour. If the newly calculated cumulative PIE is less than the previous total (En+1<En), the connection is extended in the same direction. This connection expansion ceases when the addition of a neighboring grid point's PIE results in a higher cumulative PIE than the preceding total.
   The above-described exercise is repeated for each grid point in all directions of the gridpoint.
   Through this systematic and multidirectional expansion, an intricate network of grid points, termed a “patch,” is formed which is a collection of all gridpoints extended from one gridpoint.
   Within this patch, the grid point exhibiting the lowest PIE is identified. A region with a radius of 3.5 Å around this grid point is then delineated.
   All the grid points within this region are compiled, and the sequence of probes at these points are noted from the N-terminal to the C-terminal direction of a specific protein in the grid. This sequence of probes is referred to as internal database probe pattern.
   The above process is extended to every grid point that falls within 1 Å of any amino acid of the protein
   The outcome of this expansive procedure is the formation of several patches and corresponding internal database probe patterns, derived from all grid points that is within 1 Å of any amino acid of the protein
   If a patch is large and leaves a significant portion of the protein untouched within the 3.5 Å radius region, a second grid point having the next lowest PIE in the same patch is selected, and another 3.5 Å region is chosen to create a second probe pattern from the same patch.
   This process is repeated until the maximum coverage of the patch is achieved, obtaining all possible probe patterns of that patch.
This process is carried out for every protein in the internal database, resulting in several internal database probe patterns derived from all the proteins in the database

### Alignment Algorithm

The algorithm looks at the problem of sequence database search, wherein we have a query pattern, which is a string of probes, and a template patterns(s), which are the strings of probes in the internal probe pattern database. The objective is to identify template probe patterns that are similar to the query pattern. The algorithm identifies an initial match and fine-tunes it to find a good alignment which meets a threshold score.

The steps are as follows:
1. Split query patterns into overlapping short segment of length L
2. The query pattern is first split by looking at all substrings of L consecutive probes in the query. To find the similar short segments these short segments are modified slightly and their similarity to the original sequence is computed. More dissimilar short segments are progressively generated until the similarity measure drops below a threshold T. This affords flexibility to find matches that may not have exactly L consecutive matching characters in a row, but which will have enough matches to be considered similar, to meet the threshold score.
3. Find all possible similar short segments for each short segment
4. Locate each short segment in the database, as to where each short segment occurs in any pattern. Call these the starters, and let S be the collection of starters.
5. Then, all of these short segments are used to find seeds of L consecutive matching probes.
6. Extend the starters in S until the score of the alignment drops off below some threshold X.
7. Report matches with overall highest alignment scores.

Extend these seeds to find an alignment using the local alignment method, until the score drops below a certain threshold X. Since the region we are considering is a much shorter segment, this will be achieved faster.

The algorithm is such that when L is big, there are fewer spurious hits/collisions, thus making it faster, but there are disadvantages that, the short segments tend to grow bigger and only few hits may come up. On the other hand, if L is too small, too many hits will be returned which will impact the extension and alignment step consuming more time.

If T is higher, the algorithm will be faster, but there are possibilities that evolutionarily distant sequences may not be identified.

The value of X determines the sensitivity of the algorithm. A stringent X value, despite less stringent L and T, will result in trying unnecessary sequences that would not meet the stringency of X thus increasing the computation time.

Overall, the algorithm works similar to BLAST algorithm, however the algorithm focusses on finding closest to farthest matches, without gap in the alignments. The alignments are scored using a scoring matrix similar to PAM, wherein every probe is scored against itself and every other probe.

On finding all the possible matches, the alignments ranked from highest to lowest, however a template (aligned internal database probe pattern) is considered the best only if its total PIE is lesser than that of the query, if not the algorithm moves to the next high scored alignment.
Example of Alignment Matrix

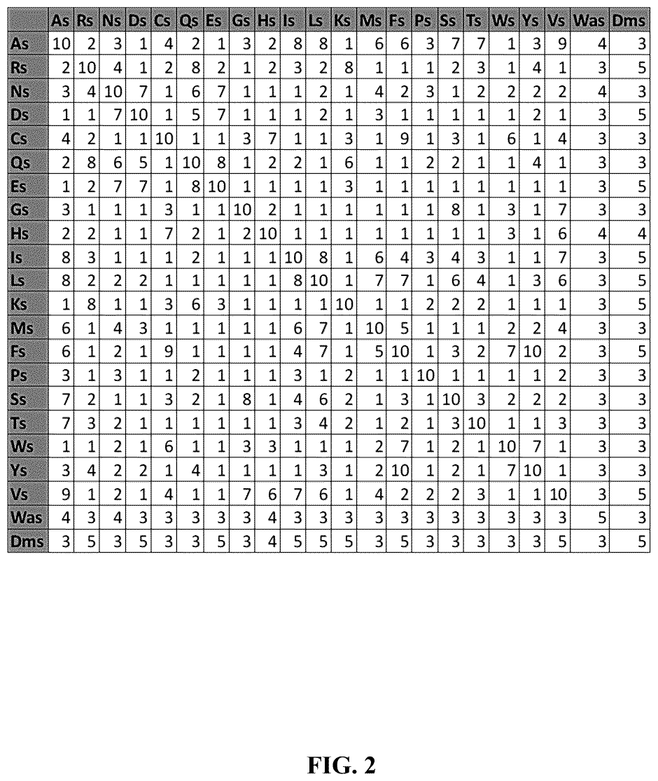

FIG. 2 shows the 22 probes are scored against one another in the matrix which is used for scoring alignment of query probe pattern and internal database probe pattern. These matrices are typically constructed based on large datasets of aligned protein sequences. The idea is to calculate how often specific amino acid substitutions occur in evolutionarily related proteins. Each cell in the matrix represents a score or penalty for substituting one amino acid with another. The score can be positive (indicating similarity) or negative (indicating dissimilarity). The higher the score, the more similar the amino acids are considered. The diagonal cells represent matches between identical amino acids. These cells typically have the highest positive scores, as identical amino acids are considered highly similar. The off-diagonal cells represent substitutions between different amino acids. The values in these cells are based on the frequency of observed substitutions in the dataset used to construct the matrix. If a substitution is common, it will have a higher positive score, indicating similarity. If it is rare, it may have a lower score or even a negative score, indicating dissimilarity. To score an alignment between two protein sequences, you traverse the alignment and look up the scores for each pair of aligned amino acids and apply gap penalties for any gaps introduced. The total alignment score is the sum of these individual scores.

### DETAILED DESCRIPTION OF THE INVENTION

The present invention is a method to engineer proteins in the following manner
a) The protein of interest is placed in a 3D grid made of grid points spaced 1 Å apart (or 2 Å apart)
b) Within the gridspace, at every gridpoint variety of probes are placed iteratively where each probe molecule simulates the interaction characteristics of an amino acid or a solvent molecule with the protein that is enclosed in the grid. The probe is placed such that, the geometric center of mass of the probe aligns with the center of the designated grid points. This ensures a standardized and consistent placement of the probes within the system, facilitating a systematic exploration of potential interactions across the entire grid.

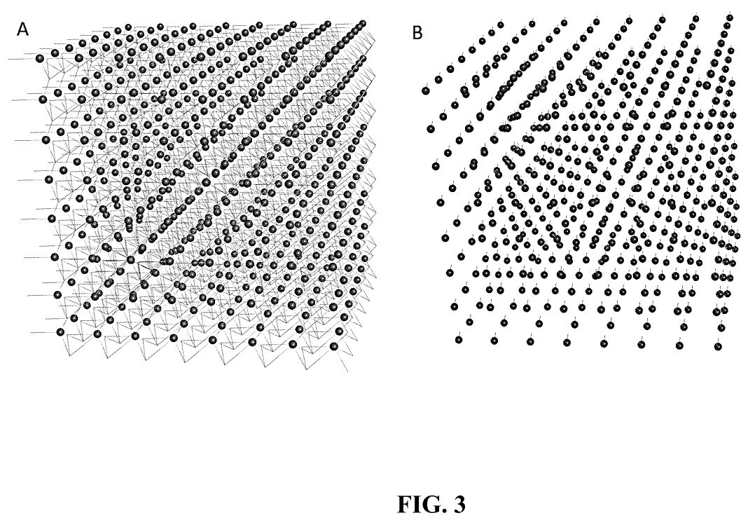

The Grid of Alanine Side chain (As) is shown in FIG. 3. (A). This grid comprises probes positioned at each grid point by aligning the geometric center of the probes to each grid point. The Grid of Water (Was) is shown in FIG. 3. (B). This grid follows the same configuration method as the Alanine grid. The probes in each grid are fine-tuned to accommodate the protein's structure as elaborated in the detailed description. Fragment Molecular Orbital energy and pair interaction energy are computed for each amino acid present in the protein and for each probe individually. The calculated energy is then registered at the corresponding grid point.”
c) The PIEs are calculated for each interaction across all grids, resulting in a comprehensive interaction profile for the protein. PIE between the probe molecules and the protein's amino acids within the grid is calculated using the Fragment Molecular Orbital technique; The probes are rotated to explore possible interactions with the protein of interest in the grid, however masked from one another to avoid inter-probe interactions The van der Waals (VDW) radius of the probe is adjusted to facilitate interactions, and the interaction energies are calculated using Pair Interaction Energy Decomposition Analysis (PIEDA) of the Fragment Molecular Orbital method. The energy of interaction is then stored at the respective grid points.
d) The PIEs are calculated for each interaction across all grids, resulting in a comprehensive interaction profile for the protein.
   Below is an example of PIE calculated, when an Alanine side chain probe (As) interacts with certain amino acids.

TABLE 1

| | | | | CT + J | | | PCM(sol) |
|---|---|---|---|---|---|---|---|
| | | | | (Charge | | | (Polarizable |
| | | Pair | | Transfer | | | Continuum |
| Residue | ALA (probe) | interaction | ES | and | DI | Ex | Model |
| of the | coordinates | energy | (Electrostatic | Polarization | (Dispersion | (Exchange | Solvation |
| protein | (X, Y, Z) | (kcal/mol) | Interaction) | Energy) | Interaction) | Interaction) | Energy) |
| MET1 | 18.224 | −7 | −2.7 | −1.6 | −0.4 | 0.5 | −2.8 |
| | −5.735 | | | | | | |
| | 2.388 | | | | | | |
| Leu2 | 22.409 | −8.8 | −3.5 | −2.1 | −0.9 | 0.3 | −2.6 |
| | −4.175 | | | | | | |
| | 3.413 | | | | | | |
| VAL3 | 22.627 | 0.2 | −1.2 | −0.7 | 0.4 | 2.2 | −0.5 |
| | 1.600 | | | | | | |
| | 0.216 | | | | | | |
| Nth AA | X, Y, Z | — | — | — | — | — | — |

The table shows the positional coordinates and calculated pair interaction energy values of Alanine (ALA) probes relative to specific amino acids in the protein. The first column lists the amino acids of the protein by their abbreviated names and sequence number. The second column provides the three-dimensional coordinates (X, Y, Z) of the As probe corresponding to each amino acid in the protein inserted in the Grid. The third column presents the pair interaction energy value between each listed amino acid and its corresponding ALA probe, calculated as described in the methodology. An example row reads: ‘MET1, (18.224, −5.735, 2.388), −4.56’, indicating that the MET1 amino acid has an ALA probe at coordinates (18.224, −5.735, 2.388) with a pair interaction energy value of −4.56.”

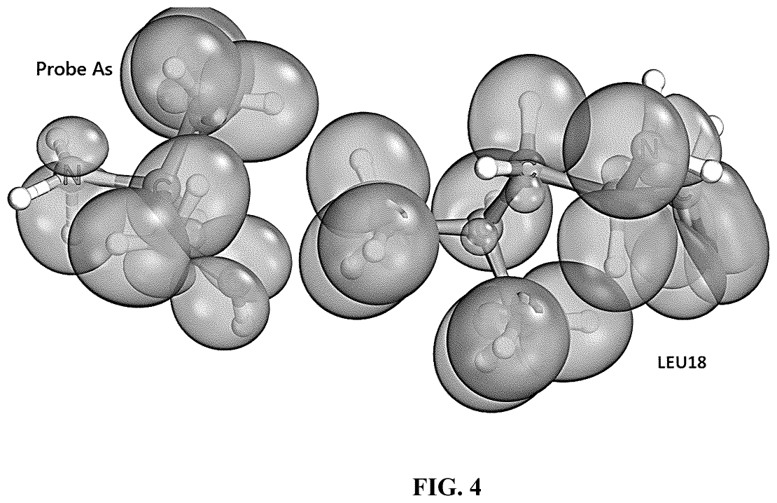

FIG. 4 shows an example of Fragment Localized Molecular Orbitals and Pair Interaction Energy Calculation; figure showing the fragment localised molecular orbitals of Alanine side chain probe (As) and that of the amino acid Leucine interacting from which PIE is calculated. Figure. 4 illustrates the fragment localized molecular orbitals involved in the interaction between LEU18 of an enzyme and the probe As. These molecular orbitals represent the electronic structure and play a key role in determining the nature of the interaction. The calculated pair interaction energy between LEU18 and As is −6.6 kcal/mol, indicating a strong attractive interaction. The PIE is composed of various terms: ES (Electrostatic Interaction) −2.1 kcal/mol, CT+J (Charge Transfer and Polarization Energy) −1.2 kcal/mol, DI (Dispersion Interaction) −0.8 kcal/mol, Ex (Exchange Interaction) 0.1 kcal/mol, and PCM (sol) (Polarizable Continuum Model Solvation Energy) −2.6 kcal/mol. These terms represent specific energy contributions to the overall PIE, highlighting the various forces involved in the interaction.
e) Each grid point stores energy details and the probe type that exhibited the least PIE during interactions with the protein's amino acids
f) Subsequently, the grid now comprises of mixed probe types based on the ones that exhibited the least PIE on interaction with the amino acids of the protein in the grid.

For example, it may be that aminoacid 23 of a protein shows the least PIE on interacting with the probe type As, among all the probes in the grid and amino acid 24 shows the least PIE on interacting with the probe type Fs, among all the probes, the new grid will have probe type As on the gridpoint interacting with amino acid 23 and the probe type Fs on the gridpoint interacting with amino acid 24.

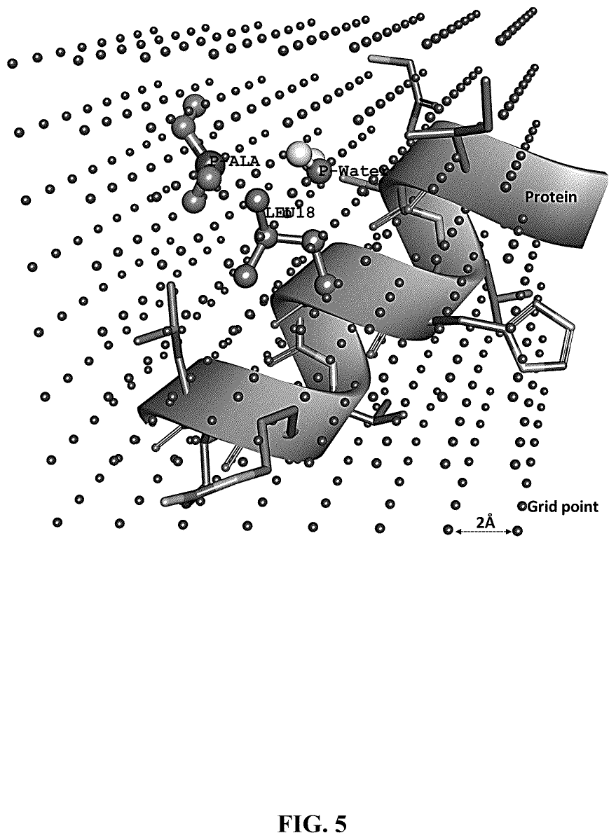

FIG. 5. illustrates a section of the enzyme, ensconced within a grid populated by grid points and probes placed 2 Angstroms apart. For clarity and convenience, only two types of probes are displayed from a mixed grid: one representative of water and the other symbolizing Alanine. These probes are strategically located close to Leucine 18 and their positions are determined based on the Fragment Molecular Orbital and pair interaction energies calculations between Leucine 18 and the other amino acids. In this particular scenario, the geometric center of the probes is situated on the grid. The probes are then fine-tuned to achieve a Vanderwaals fit with the protein sidechain.
g) The computational process is extended by systematically augmenting the sum of the Pair Interaction Energy stored on each grid point, on the negative scale between each grid point and its neighboring grid points within the constructed three-dimensional grid space encapsulating the protein structure. This methodical progression persists until the integration of an additional grid point leads to a shift in the cumulative PIE towards the positive scale, signaling a less favorable interaction.
h) Extension is done on every gridpoint, the extension can be explained as, a grid point being connected to its neighbour and the PIE stored on the neighbour is added to its PIE, if the cumulative PIE is lesser than the last PIE, then the connection grows to the next neighbour. If the newly calculated cumulative PIE is less than the previous total (Σn+1<Σn), the connection is extended in the same direction. This connection expansion ceases when the addition of a neighboring grid point's PIE results in a higher cumulative PIE than the preceding total.
i) The above-described exercise is repeated for each grid point in all directions of the gridpoint.
j) Through this systematic and multidirectional expansion, an intricate network of grid points, termed a “patch,” is formed which is a collection of all gridpoints extended from one gridpoint.
k) The invention extends the process mentioned above to every grid point that falls within 1 Å of any amino acid of the protein. This exhaustive coverage of the protein's geometry allows the comprehensive mapping of the grid.
l) The outcome of this expansive procedure is the formation of several patches.

For example, if 10,000 grid points are within 1 Å of any amino acid of the protein within the grid, this will lead to the creation of 10,000 individual 27 grid point spaces, and correspondingly, 10,000 patches
m) Following the generation of these patches, they are organized in ascending order based on their size, from smallest to largest. This order is crucial as the smallest patch, indicating a region with minimal extension due to an increase in cumulative PIE upon connection to a neighboring grid point, suggests a region of instability or unfavourable interactions within the protein. Thus, the investigation begins with the smallest patch.

n) Within this patch, the grid point exhibiting the highest PIE is identified, a region with a radius ranging from 3.5 Å to 5 Å around this gridpoint is then delineated. All the grid points within this region are compiled, and the sequence of probes at these points are noted from the N-terminal to the C-terminal direction of the protein. This sequence of probes is referred to as a query probe pattern.
o) The query probe pattern derived from the smallest patch is then compared against the patterns within the internal database using the alignment algorithm. For instance, within the 3.5 Å radius of the smallest patch, if the sequence of probes connected in the N to C terminal direction of the protein is, Fs, Gs, Rs, Ts, Hs, and Ms, the aminoacids of the protein to which these probes are interacting form the sequence of amino acids corresponding to this query probe pattern in the N to C terminal direction.

Further explaining the same, it would be that if the probe Fs interacts with the amino acid W, if Gs interacts with amino acid A, if Rs interacts with H, if Ts interacts with S, if Hs interacts with R, and if Ms interacts with V, then for the query probe pattern FsGsRsTsHsMs the corresponding aminoacid sequence will be WAHS RV.

In the given example query probe pattern FsGsRsTsRsMs, if Rs is the probe with the highest PIE, it is designated as the hotspot.

This query probe pattern is then matched against the internal database probe patterns using the alignment algorithm described earlier. This operation may return numerous hits based on alignment match. These hits are then sorted based on the total PIE energy of the probe pattern, subsequently ranking the hits based on the total PIE energy of the probe pattern; however, all the matching internal database probe patterns are used for mutating the query probe pattern, starting with the highest-ranking internal database probe pattern

If the example Query Probe pattern, FsGsRsTsHsMs, has total PIE of −12 kcal/mol and searching the same against the internal database returned three matches with total PIE as given below

FsGsRsTsRsMs−Total PIE=−23 kcal/mol Database Probe pattern 1:

FsAsRsSsHsMs−Total PIE=−19 kcal/mol Database Probe pattern 2:

WsGsRsTsHsCs−Total PIE=−18 kcal/mol Database Probe pattern 3:

All the three internal databse probe patterns will be used to mutate the query, starting with the one with the least total PIE, in this illustration, it will be database probe pattern 1 with total PIE −23 kcal/mol

In the database probe pattern-1 FsGsRsTsRsMs, if the probe Fs interacts with the amino acid Y, if Gs interacts with amino acid V, if Rs interacts with R, if Ts interacts with S, if Rs interacts with N, and if Ms interacts with V, then for the database probe pattern FsGsRsTsRsMs the corresponding aminoacid sequence will be YVRSNV

| | | | | | | |
|---|---|---|---|---|---|---|
| Amino acids connected to | W | A | H | S | R | V |
| Query Probe pattern: | | | | | | |
| Query Probe pattern: | Fs | Gs | Rs | Ts | Hs | Ms |
| Database probe pattern 1: | Fs | Gs | Rs | Ts | Rs | Ms |
| Amino acids connected to | Y | V | R | S | N | V |
| Database Probe pattern 1: | | | | | | |

In this instance, Rs is the probe with the highest PIE and hence, serves as the hotspot on the Query Probe Pattern. The alignment matches Database Probe pattern 1, wherein an identical probe is aligned with the hotspot. The amino acid corresponding to the hotspot Rs of the Query Probe is H, while in the Database Probe Pattern 1, it is R. Consequently, in the query protein (the protein of interest), the amino acid H is mutated to R if the PIE of the aligned probe from the database is less than that of the hotspot probe on the query.
p) Following this first mutation, in the same query probe pattern a second position is also chosen for mutation. However, the criteria for this selection differs: it's based on a probe in the database probe pattern 1, in the alignment which has the least PIE and is lesser than that of the query probe. The corresponding amino acid is then used to mutate the respective query probe's corresponding amino acid, leading to two mutations in this region of the protein. In the event that the PIE of the aligned probe from the database is greater than that of the hotspot probe on this query pattern, that position is not mutated. Instead, the next highest energy query probe is mutated with the corresponding amino acid of the database probe aligned with it, thus making a variant with single or double mutations.
   In the entire process for a database probe pattern to be considered as a matching internal database probe pattern, the qualifying criteria are, a 99% alignment match and a total PIE less than that of the query probe pattern. Only the matching internal database probes, are selected. If 300 probes meet this criteria, 300 single or double variants with corresponding amino acids of internal probe pattern on the query probe are generated.
q) With this first set of mutations, each of these variants is subjected to local geometry optimizations or short molecular dynamics simulation.
r) Followed by this, steps a to m are repeated to identifying patches, however the procedure is now executed on a mutated protein and is restricted to regions corresponding to the smallest patch and the patches that interface with the smallest patch obtained from the previous steps
s) This process may produce patches, larger than the original smallest patch.
   This step is performed for every variant, observing the growth of the patches from the smallest original patch.
t) The variant that caused the most substantial growth from the original small patch confirms as the first set of mutations in the protein of interest.
u) Following this, the second smallest patch is selected, and the process (Steps n to s) is repeated, leading to the second set of mutations. This process is carried through the third and fourth smallest patches as well, producing the third and fourth set of mutations in the protein of interest respectively.
v) By the end of this multi-step process, a final variant of the protein, with 2 to 8 mutations, is generated. This process is highly adaptable and can be localized to any part of the protein, be it the active site interacting with the substrate, intermediate or product or a specific region for the identification and optimization of patches.

### EXPERIMENTAL DATA

The enzyme tyrosine hydroxylase was engineered using this method. The method described here is used to incorporate mutations and activity of the engineered enzymes was predicted

### Conversion of L-Tyrosine to L-DOPA by Tyrosine Hydroxylase

L-DOPA (L-3,4-dihydroxyphenylalanine) is a precursor molecule in the biosynthesis of several important neurotransmitters, including dopamine, norepinephrine, and epinephrine. The conversion of L-Tyrosine to L-DOPA is a crucial step in this biosynthetic pathway, and it is catalyzed by the enzyme Tyrosine Hydroxylase (TH).

Enzyme Tyrosine Hydroxylase (TH): Tyrosine Hydroxylase is an enzyme that plays a vital role in the synthesis of catecholamines, which are a class of neurotransmitters. It is primarily found in nerve cells of the central nervous system (CNS) and the peripheral sympathetic nervous system. TH is a rate-limiting enzyme, meaning that its activity controls the overall rate of catecholamine biosynthesis.

Mechanism of Conversion: The conversion of L-Tyrosine to L-DOPA involves the hydroxylation of the tyrosine molecule at the para position of the phenyl ring. This hydroxylation reaction introduces a hydroxyl (—OH) group onto the aromatic ring of the tyrosine molecule.

The reaction requires molecular oxygen (O₂) as a co-substrate and tetrahydrobiopterin (BH4) as a cofactor. The hydroxylation of tyrosine is a complex biochemical process that involves the activation of the enzyme, binding of substrates, and several intermediate steps. The iron atom in the active site of Tyrosine Hydroxylase plays a critical role in facilitating the hydroxylation reaction.

Importance of L-DOPA: L-DOPA serves as a critical precursor for the subsequent synthesis of dopamine, a neurotransmitter that plays a significant role in mood regulation, motor control, and various physiological processes. Additionally, L-DOPA has medical significance as it is used in the treatment of Parkinson's disease. Parkinson's disease is characterized by a deficiency of dopamine due to the degeneration of dopamine-producing neurons. L-DOPA can cross the blood-brain barrier and be converted into dopamine, helping alleviate some of the motor symptoms associated with the disease.

The conversion of L-Tyrosine to L-DOPA by Tyrosine Hydroxylase is a pivotal step in the biosynthesis of neurotransmitters like dopamine. This enzymatic process is vital for normal neurological function and has implications for both basic neuroscience research and medical applications in conditions like Parkinson's disease.

The enzyme reaction catalyzed by Tyrosine Hydroxylase (TH) has important commercial applications, particularly in the pharmaceutical and biotechnology industries. Here are a few notable applications:
1. Production of L-DOPA for Parkinson's Disease Treatment: One of the most significant applications of the conversion of L-Tyrosine to L-DOPA is in the pharmaceutical industry for the production of L-DOPA. L-DOPA is a primary medication used to treat the symptoms of Parkinson's disease. Commercially, L-DOPA is synthesized through fermentation processes or chemical synthesis, and it plays a vital role in improving the quality of life for individuals with Parkinson's disease.
2. Neurotransmitter and Drug Research: The study of enzymes like Tyrosine Hydroxylase has direct applications in understanding neurotransmitter synthesis and regulation. Pharmaceutical companies and researchers study these enzymes to develop drugs that target specific steps in neurotransmitter synthesis pathways. Such drugs can have applications in treating various neurological and psychiatric disorders.
3. Biocatalysis and Enzyme Engineering: Enzymes like Tyrosine Hydroxylase have the potential to be used as biocatalysts in various industrial processes. Researchers are interested in enzyme engineering techniques to modify and optimize these enzymes for specific reactions. This could lead to the development of more efficient and eco-friendly methods for producing certain chemicals or pharmaceutical compounds.
4. Biopharmaceutical Production: Biopharmaceuticals, which include a wide range of therapeutic proteins and enzymes, are produced using recombinant DNA technology. The expression of enzymes like Tyrosine Hydroxylase can be engineered into cells to produce therapeutic proteins that are essential for various medical treatments.
5. Biotechnology and Neuroscience Research: Enzymatic reactions involving Tyrosine Hydroxylase are fundamental to the understanding of neurotransmitter synthesis and the regulation of neural pathways. Researchers in biotechnology and neuroscience use these reactions as models to investigate various aspects of cellular function, neurotransmission, and neurodegenerative disorders.

While the enzyme Tyrosine Hydroxylase have the potential to be used as biocatalysts in industrial processes, the full optimization of their industrial applications is an ongoing area of research and development. While some progress has been made, there's still much to explore and improve upon in terms of their efficiency, specificity, stability, and scalability for various applications.

The method described here was used to modify a TH to enhance the factors given below including catalytic activity, substrate specificity, and stability under industrial conditions. These modifications can enable enzymes to perform reactions more efficiently, at higher yields, and in an eco-friendlier manner compared to traditional chemical methods.
1. Specificity: Ensuring that the enzyme catalyzes the desired reaction with high specificity and minimal side reactions.
2. Stability: Making the enzyme stable over a range of temperatures, pH levels, and other environmental conditions encountered in industrial processes.
3. Yield: Increasing the yield of the target product to make the process economically viable.
4. Scale-up: Demonstrating that the enzyme works efficiently at larger scales, which is crucial for industrial production.
5. Cost efficiency: Evaluating the overall cost of enzyme production, purification, and implementation in comparison to alternative methods.
6. Integration: Integrating the enzyme-based process into existing industrial workflows without disrupting efficiency.
7. Eco-Friendliness: Demonstrating that the enzyme-based process has environmental benefits over traditional chemical methods, such as reduced waste and energy consumption.

While there are successful examples of using enzymes in various industrial processes, there is ongoing research to optimize their application in different sectors, ranging from pharmaceuticals and fine chemicals to biofuels and food production. The optimization process may involve iterative cycles of experimentation, analysis, and refinement to achieve the desired outcomes. TH is one example that is explained herewith.

### Expression Protocol for Tyrosine Hydroxylase Enzyme Variants

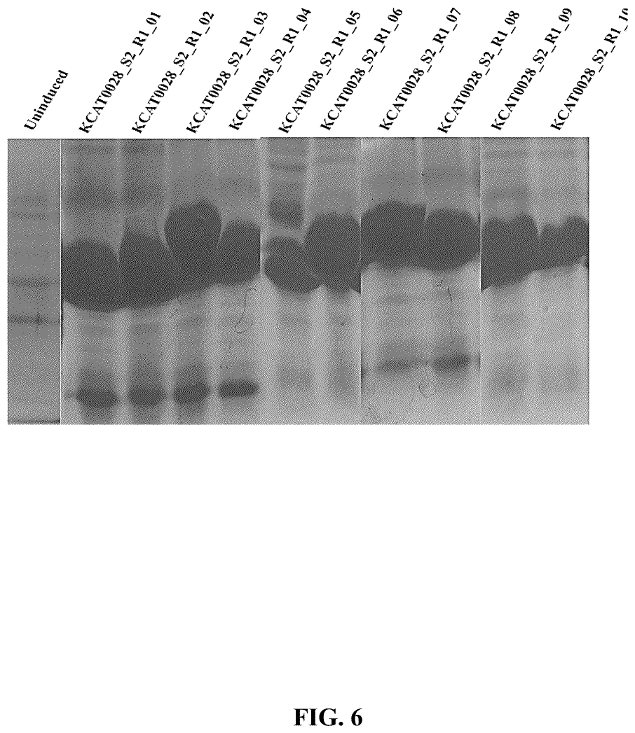

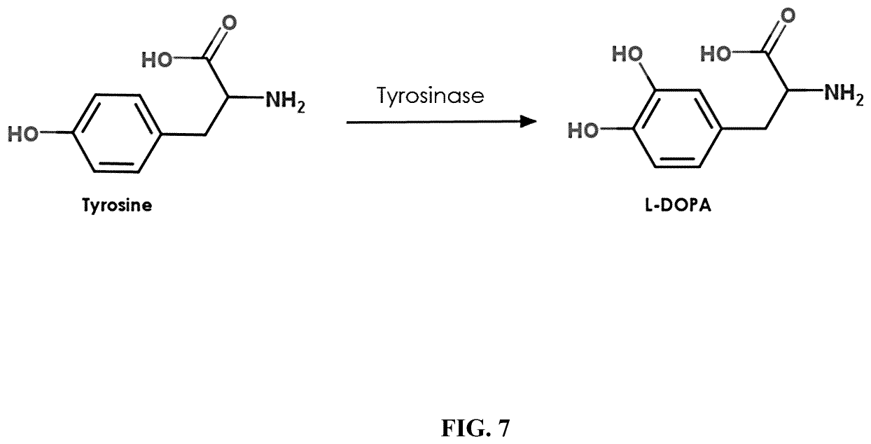

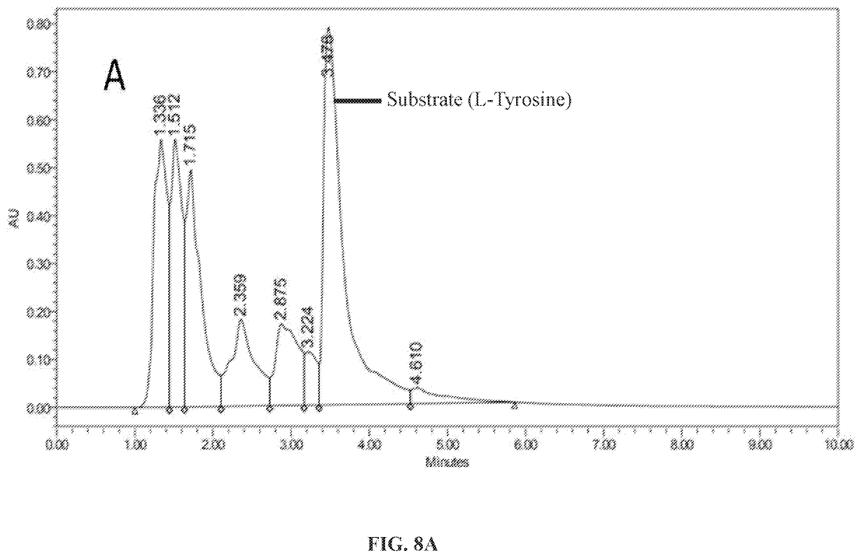

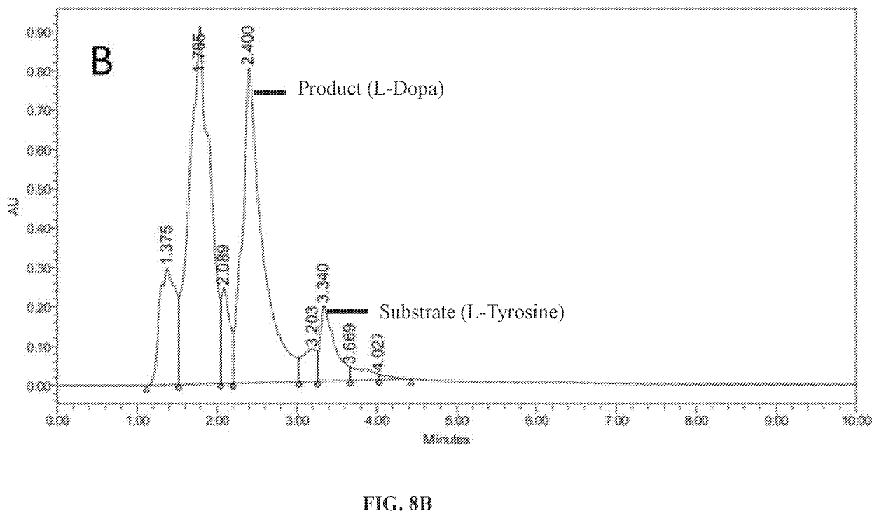

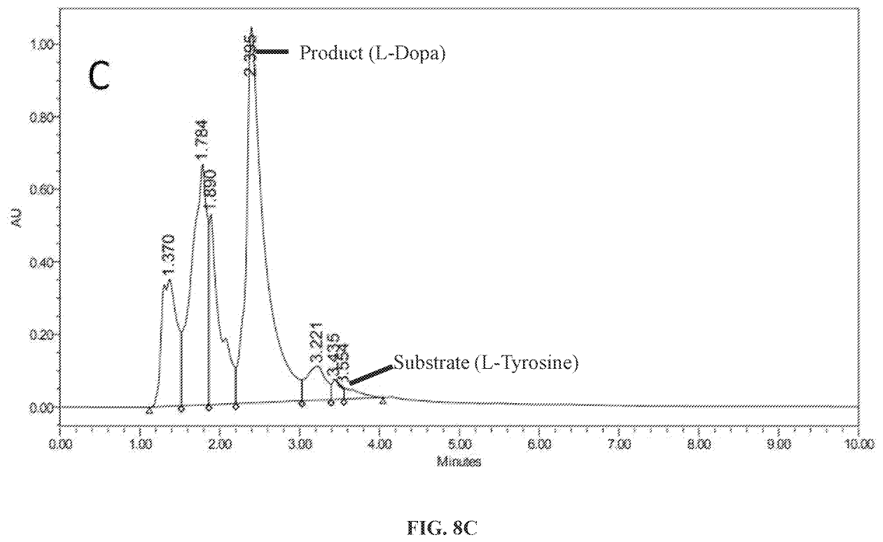

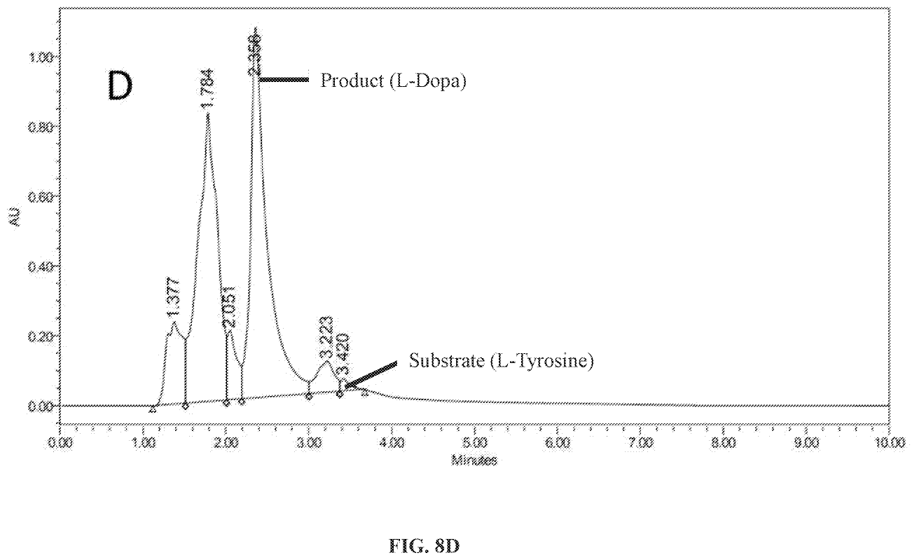

The recombinant plasmid carrying the gene of interest was introduced into BL21 competent cells through a transformation process. This genetic manipulation ensured that the cells gained the ability to produce the desired enzyme. After the transformation, the cells were cultivated in Luria Bertani (LB) broth. This growth medium provided the necessary nutrients for the cells to thrive. Kanamycin, an antibiotic, was added to the medium at a concentration of 50 μg/ml. This served the purpose of selecting cells that successfully incorporated the recombinant plasmid, as only those cells could withstand the antibiotic. The production of the enzyme was induced at a specific point in the cell growth process. When the optical density (OD) of the culture reached 0.6 at a wavelength of 600 nm (OD 600 nm), indicating a certain cell density, 0.5 mM of isopropyl-β-D-thiogalactoside (IPTG) was added to the culture. IPTG is an inducer that mimics lactose and triggers the expression of the target gene. Its addition prompts the cells to start producing the enzyme. Following IPTG induction, the culture was shifted to a lower temperature of 25° C. This temperature change was intended to optimize enzyme production. The culture was allowed to incubate for a duration of 16 hours. This extended period allowed sufficient time for the cells to synthesize and accumulate the enzyme of interest. After the incubation, the culture was collected for further processing. The cells were harvested by subjecting the culture to centrifugation at 4000 rpm for 15 minutes. This centrifugation step separated the cells from the growth medium, resulting in a cell pellet. The harvested cells were subjected to lysis, a process that breaks open the cell membranes to release the cellular contents. To initiate lysis, 1 mg/ml of lysozyme was added to the cells. The lysozyme enzyme helps weaken the cell walls. The cell suspension was then resuspended in a buffer containing 50 mM Tris-HCl, 150 mM NaCl, 1 mM CaCl2, and 1 mM KCl, all adjusted to a pH of 8.0. Sonication, a technique that uses sound waves, was applied to further break down the cell membranes and release the cellular proteins. After sonication, the cell lysate was subjected to centrifugation at 8000 rpm for 15 minutes at 4° C. This centrifugation step separated the crude protein lysate into different fractions. The soluble protein fraction, which contained the enzyme of interest, was obtained. The obtained crude protein lysates were analyzed using a technique called sodium dodecyl sulfate polyacrylamide gel electrophoresis (SDS-PAGE) FIG. 6. The engineered tyrosine hydroxylase polypeptide is capable of converting L-Tyrosine to L-DOPA as is the reaction of interest shown in Figure. 7.

The engineered tyrosine hydroxylase polypeptide can catalyse this conversion with an activity that is equal to or greater than 80%, in industrial reaction conditions at temperature range of 20-55° C. and pH range of 4-9.

The engineered tyrosine hydroxylase can be expressed in bacterial host cell like E. coli, using the expression vector pET28a(+) on which the engineered tyrosine hydroxylase polynucleotide is constructed and cloned to the bacteria and cultured in a suitable medium. The host cells can be used for the expression and isolation of the engineered tyrosine hydroxylase enzymes or alternatively, they can be used directly for the conversion of L-Tyrosine to L-DOPA.

In another aspect, the engineered tyrosine hydroxylase, in the form of whole cells, crude extracts, isolated polypeptides, or purified polypeptides, can be used individually or as a combination of different engineered tyrosine hydroxylase.

### Assay Conditions for Tyrosine Hydroxylase Activity

Experiments were conducted using a 1 ml reaction volume within a phosphate buffer solution of 50 mM strength and a pH of 7.0. The reaction buffer was supplemented with key components to optimize reaction kinetics. The mixture contained L-Tyrosine at a concentration of 2.5 mM, serving as the substrate. Ascorbic acid at 5 mM, employed as a reducing agent and enzyme inhibitor. Ascorbic acid served as a safeguard against diphenolase activity by inhibiting undesirable conversions. Copper ions (1 μM) were introduced as cofactors to facilitate the reaction process. The reaction was initiated by introducing free tyrosinases into the mixture, acting as enzymatic catalysts. To maintain equilibrium, additional ascorbic acid (5 mM) was added to ensure proper directionality of the reaction. The reaction mixture was placed in an incubator shaker operating at 45° C. with constant agitation (200 rpm). Adequate oxygen supply was ensured to support enzymatic activity and maintain reaction stability. The reaction was allowed to proceed for a precisely timed interval of 60 minutes, optimizing reaction progression. The conversion of L-tyrosine to L-DOPA was successfully achieved.

### HPLC for L-DOPA Estimation:

High-performance liquid chromatography (HPLC) analysis was executed on a Waters HPLC instrument, specifically employing a C18 Zorbax column (150×4.6 mm, 5 μm) paired with a UV detector set at 280 nm, a wavelength where L-DOPA displays substantial UV absorbance. The chosen mobile phase for this process was a blend of 0.1 N acetic acid and methanol in a 10:1 ratio, maintained at a flow rate of 1.0 mL/min for a duration of 10 minutes at a temperature of 30° C. An injection volume of 20 μL was utilized. The sample, which contained L-DOPA, was curated from a specific reaction under designated conditions. This sample was then set up within the HPLC system, which was pre-configured with the earlier mentioned column and connected to the mobile phase reservoirs, typically within a flow rate window of 1.0 to 1.5 ml/min.

To ensure accurate quantification of L-DOPA, a calibration curve was constructed. This involved preparing a sequence of L-DOPA standard solutions across an anticipated concentration range. These were subsequently introduced into the HPLC system, and their chromatograms were documented. Leveraging the peak areas obtained from these standards, a calibration curve, showcasing the relationship between concentration and peak area, was formulated. Once the sample containing L-DOPA was injected, the mobile phase journeyed through the column, segregating L-DOPA from potential contaminants. This separation stemmed from varying interaction intensities between these compounds and the column's stationary phase. As compounds exited the column, their absorbance was gauged by the UV detector. To finalize the process, the peak area of the L-DOPA in the resultant chromatogram was measured. The calibration curve's equation facilitated the transformation of this peak area into an actual L-DOPA concentration. All analytical outcomes, inclusive of the deciphered L-DOPA concentration in the sample, were then systematically compiled and reported.

### Results of L-DOPA Estimation

The HPLC chromatograms depicted in FIGS. 8A-8D offer an insightful representation of the enzymatic conversion of L-Tyrosine to L-DOPA. This visual distinction clearly delineates the activity of A) the wild-type enzyme against the performances of the engineered enzyme variants shown in B, C, and D. The assay, designed to emulate optimal catalytic conditions for these enzymes, was executed under the following parameters: a substrate (Tyrosine) concentration of 10 mg/ml; an alkaline environment with a pH of 9.5; a temperature maintained at 45° C.; and an enzyme concentration of 0.1 mg/ml. The entire reaction spaned a 45-minute timeframe.

From the chromatograms, an evident observation emerges: the wild enzyme largely remains dormant, presenting no discernible activity within the given conditions. In contrast, the engineered variants demonstrated substantial conversion. This conspicuous difference emphasizes the advancements achieved through the engineering process. Notably, the enzyme load was just 1% for all the complete conversion by the engineered enzymes. This observation is particularly promising, highlighting the efficacy of the engineered enzymes and hinting at potential cost savings in large-scale applications.

TABLE 2
Enzymatic Activity of Engineered Variants at 45° C. and pH 9.5

| Variant ID | % conversion (L-DOPA) |
|---|---|
| Wild | 0% |
| Mutant 001 | 85% |
| Mutant 002 | 89% |
| 6. Mutant 003 | 7. 91% |

Table 2. presents a comparison of the conversion rates for L-Dopa by various enzyme variants. These variants were engineered using the described technology.
Variant ID: Refers to the identification tag assigned to each protein variant, including the wild type (original, non-mutated enzyme) and the three mutants.
% conversion (L-Dopa): Denotes the efficiency of each enzyme variant in converting their respective substrates to L-Dopa. The conversion rates are expressed in percentages.

Key insights from the table:
1. The wild type enzyme exhibited no conversion, registering at 0%.
2. The engineered mutants (Mutant 001, Mutant 002, and Mutant 003) showcased significantly higher conversion rates, ranging from 85% to 91%, underscoring the effectiveness of the engineering technology.
   The testing conditions maintained for assessing the enzymatic activity were a temperature of 45° C. and a pH of 9.5.

### Advantages of the Invention

The present invention introduces a novel and robust method to engineer proteins using a unique probe-grid system and a comprehensive evaluation of Pair Interaction Energy in the context of an array of probe types. This innovation confers several advantages as described below. The method enables a systematic and extensive exploration of potential interactions between the protein of interest and various types of probes within the 3D grid system. The probes are methodically positioned, and their interactions with the protein residues are independently evaluated, thus providing a comprehensive understanding of the protein-probe interactions. The grid-based system and the detailed analysis of PIE allow for high precision in the investigation of protein-ligand interactions. The iterative process of probe positioning and PIE calculation significantly increases the accuracy of predicting possible interaction sites and the effectiveness of potential mutations. The invention improves the predictability of successful protein engineering. By thoroughly mapping the interactions between different probe types and protein residues, this method can anticipate how certain mutations may influence the protein's function. The interaction data stored in the internal database further enhance this predictive power. The technique is applicable to any protein, thus providing broad applicability in protein engineering. Furthermore, the methodology can accommodate different probe types, facilitating a diverse range of studies and applications. By identifying the grid points with the least PIE, the methodology effectively pinpoints the most optimal positions for mutations. This aids in devising effective strategies for protein engineering, enabling more targeted and successful modifications. The use of a mixed grid—combining the most effective probes from individual grids-streamlines the protein engineering process. The ability to target specific regions of the protein for study reduces unnecessary calculations and optimizes the computational efficiency of the method. The methodology facilitates the generation of multiple protein variants with single or double mutations. These can be further evaluated for improved functionality or stability, thus accelerating the protein design and engineering process. The invention offers a comprehensive approach to protein engineering by incorporating aspects such as local geometry optimizations or short molecular dynamics simulations. This broad approach enhances the overall reliability and success rate of the engineering process.

In summary, this invention provides a robust, precise, and comprehensive methodology for protein engineering, effectively integrating systematic grid-based exploration, extensive PIE evaluation, efficient probe positioning, and strategic protein mutation. As such, it offers a significant advancement in the field of protein engineering and promises to enhance our ability to design and modify proteins for a wide array of applications.

What is claimed is:

1. A computer-implemented method for engineering proteins, the method comprising the steps of:
a. Constructing a three-dimensional grid space enclosing a protein structure with grid points at a distance of 1 Å or 2 Å;
b. Iteratively placing a variety of probe molecules within the grid space, where each probe molecule simulates the interaction characteristics of an amino acid or solvent molecules with the protein that is enclosed in the grid;
c. Calculating Pair Interaction Energy using the Fragment Molecular Orbital technique between the probe molecules and the protein's amino acids within the grid; each grid point stores energy details and the probe type that exhibited the least PIE during interactions with the protein's amino acids;
d. Extending the computational process by systematically augmenting the sum of the Pair Interaction Energy on the negative scale between each grid point and its neighboring grid points within the constructed three-dimensional grid space encapsulating a protein structure. This methodical progression persists until the integration of an additional grid point leads to a shift in the cumulative PIE towards the positive scale, signaling a less favorable interaction.
e. Subsequently, the method involves repeating this process for each grid point within the structure, taking into consideration different directional orientations, to form a network or “cluster” of grid points, herein referred to as a “patch”. This procedure is iteratively applied to all grid points within the space, thereby extending the entirety of the grid space.
f. Identifying the smallest patch among all patches, and within this smallest patch, selecting a grid point exhibiting the highest Pair Interaction Energy. The region surrounding this this grid point, with a radius ranging from 3.5 Å to 5 Å is then defined.
g. Gathering all grid points within this defined region, recording the sequence of probes at these points from the N-terminal to the C-terminal direction of the protein region. This sequence is referred to as a query probe pattern
h. Recognizing the sequence of probes such that each probe establishes a unique interaction pair with its interacting amino acid. This paired sequence forms the corresponding amino acid sequence for a given probe pattern;
i. Identifying the probe with the highest Pair Interaction Energy within the query probe pattern and designating it as the hotspot;
j. Conducting an alignment of the derived query probe pattern with the existing patterns within an internal database. This is accomplished using a predetermined alignment algorithm to locate similar internal database probe patterns;
k. Pinpointing the probe-amino acid pair in the query probe pattern that exhibits the highest Pair Interaction Energy, which is designated as the hotspot. This amino acid is then mutated to the amino acid corresponding to the probe from the matched database pattern, resulting in a modification of the original protein structure.
l. Following this modification, the method involves the repeating steps a to e to identifying patches, however the procedure is now executed on the mutated protein and is restricted to regions corresponding to the smallest patch and the patches that interface with the smallest patch
m. Repeating this process for subsequent smaller patches, leading to additional sets of mutations;
n. Concluding the multi-step process by generating a final variant of the protein with 2 to 8 mutations by combining these sets of mutations;
o. Iteratively exploring multiple such mutations for the query pattern until the smallest patch expands and grows to its maximum.

2. The computer-implemented method of claim 1, further comprising the step of selecting the probe molecules based on their ability to represent diverse molecular interaction characteristics of amino acids or solvent molecules.

3. A system for implementing the method of claim 1, localizing the process to any part of the protein, whether the active site or a specific region of protein-protein interface or any biomolecular interaction for the identification and optimization of patches.

4. The computer-implemented method of claim 1, further comprising the step of calculating the Pair Interaction Energy using the Fragment Molecular Orbital technique that encompasses a range of interaction variables between the amino acid of the protein and the probes.

5. The computer-implemented method of claim 1, further comprising the step of utilizing a predetermined alignment algorithm that aligns the derived query probe pattern with patterns in an internal database.

6. The computer-implemented method of claim 1, further comprising the step of mutating the amino acid in the probe-amino acid pair exhibiting the highest Pair Interaction Energy based on its potential contribution to the modified protein structure.

7. The computer-implemented method of claim 1, further comprising the step of generating a final protein variant prioritizing improvement in specific protein characteristics.
